(19) United States
(12) Patent Application Publication
(10) Pub. No.: US 2018/0310861 A1
(43) Pub. Date: Nov. 1, 2018

(54) WEARABLE BIOFEEDBACK BELT DEVICE

(71) Applicants: Kenneth Kushner, Madison, WI (US); Kayla Huemer, Baraboo, WI (US); Melanie Loppnow, Madison, WI (US); Anupama Bhattacharya, Verona, WI (US); Hannah Lider, Menasha, WI (US); Jessica Brand, Skokie, IL (US)

(72) Inventors: Kenneth Kushner, Madison, WI (US); Kayla Huemer, Baraboo, WI (US); Melanie Loppnow, Madison, WI (US); Anupama Bhattacharya, Verona, WI (US); Hannah Lider, Menasha, WI (US); Jessica Brand, Skokie, IL (US)

(21) Appl. No.: 15/965,901

(22) Filed: Apr. 28, 2018

Related U.S. Application Data
(60) Provisional application No. 62/492,037, filed on Apr. 28, 2017.

(51) Int. Cl. A61B 5/113; A61B 5/08; A61B 5/00

(52) U.S. Cl. CPC A61B 5/113; A61B 5/0816; A61B 5/0002; A61B 5/6823; A61B 5/7246; A61B 5/6831

(57) ABSTRACT

A wearable device for the purpose of measuring expansion of the abdominal cavity as it relates to an end-user's breathing. The device is comprised of a belt with a sensing mechanism that interfaces with an electronics box. The electronics box communicates wirelessly with a phone application, which prompts the user to choose a variety of variables so that each breathing session is customized to their breathing needs. The belt measures the physical expansion of the user's abdomen in real-time, while the application offers real-time data displayed in various engaging graphical interfaces. The belt includes a first and second inextensible belt portion with an extensible belt portion disposed between. The belt includes a belt expansion measuring device configured to measure a length of the extensible belt portion and produce an electronic signal that corresponds to the length of the extensible belt portion and thereby the expansion of the user's abdomen. The proposed device not only monitors breathing, but actively assesses breathing in an objective manner so that users can learn to improve their abdominal breathing techniques.

### CROSS-REFERENCE TO RELATED APPLICATIONS

The present application claims priority to U.S. provisional patent application No. 62/492,037, filed on Apr. 28, 2017 and entitled Wearable Biofeedback Belt Device, the entire contents of which are incorporated herein by reference.

### BACKGROUND

Abdominal breathing involves physically expanding the abdomen to its maximum position with every inhalation, and then slowly retracting the abdomen back to a comfortable resting position with each exhalation. Abdominal breathing techniques are commonly used in stress management programs and are often prescribed to patients with stress disorders and lung dysfunction. Proper abdominal breathing can be used to alleviate the effects of several maladies such as anxiety, motion sickness, asthma, chronic obstructive pulmonary disease, cardiac disease, and gastroesophageal reflux disease. General abdominal breathing is also the first step to mastering Hara breathing, which is a type of abdominal breathing tied to the Japanese art of Kyudo (Zen Archery). Both abdominal breathing and Zen meditation induce many positive physiological responses. For instance, general abdominal breathing has been correlated to overall health and mood improvements. Voluntary abdominal breathing is associated with a state of mental relaxation and an increase in whole blood serotonin levels, which corresponds to reduced anxiety. Further, low heart rate variability (HRV)—variation of the time interval between heartbeats—is associated with poor prognoses for patients with ischemic heart disease or diabetes. However, patients suffering with these conditions showed increased HRV after performing abdominal breathing. The practice of slow breathing exercises also corresponds to improved baroreflex activity, which decreases vulnerability to disorders involving autonomic hyperactivity, such as stress disorders. Additionally, a study on male smokers found that participants who practiced abdominal breathing techniques showed improved pulmonary function. Another study found that both patients with chronic obstructive pulmonary disease and healthy control subjects who practiced abdominal breathing exercises had increased lung volumes and blood oxygenation. These studies demonstrate the value of abdominal breathing both in a clinical setting and for independent users looking to improve their general wellness by reducing stress and increasing mindfulness. Further, proper abdominal breathing is important in many common activities such as singing, doing yoga, playing certain instruments, and participating in various sports.

The many benefits of proper abdominal breathing techniques are hard to attain as both abdominal breathing and Hara breathing are difficult to learn. Currently, mastering these techniques is largely contingent on the presence and subjective feedback of an instructor, making it extremely challenging to learn independently or to objectively gauge progress. Pre-existing devices are insufficient as they may solely monitor breathing without helping the user improve, and/or fail to assess the physical abdominal expansion that is key to abdominal breathing. There is therefore the need for a wearable device that actively guides the user through abdominal breathing exercises, evaluates the user's breathing technique in real-time, measures their physical abdominal expansion, and offers an objective continuum of feedback to help the user assess their performance per session and their progress over time. Such a device would be extremely beneficial for individuals in a clinical setting where abdominal breathing is an effective form of treatment, as well as for any individual looking to increase their general wellness or improve a skillset that involves proper breathing techniques.

### SUMMARY

The wearable devices disclosed herein are for users to monitor breathing and improve abdominal breathing techniques. The wearable device allows for the user to wear a belt around their abdomen which is operably coupled to a belt expansion measuring device configured to generate a signal that varies as a function of the expansion and contraction of the user's abdomen during breathing. The belt expansion measuring device further comprises a signal transmitter to transmit the signal gathered from the belt to a remote device, such as a mobile phone. The remote device can generate a breathing curve based on signals transmitted, which can be displayed to the user in a graphical form. A non-transitory computer-readable medium having stored thereon computer-readable instructions, when executed by a processor in a remote device, causes the remote device to compute a breathing curve from a signal that varies as a function of the expansion and contraction of the user's abdomen and generates a graphical display of the breathing curve.

In another aspect, the present invention is a belt comprised of a first inextensible belt portion, a second inextensible belt portion, an extensible belt portion, and a belt expansion measuring device. The extensible belt portion is disposed between the first inextensible belt portion and the second inextensible belt portion. The belt expansion measuring device is configured to measure a length of the extensible belt portion and produce an electronic signal that corresponds to the length of the belt.

In another aspect, the present invention comprises a method of monitoring breathing performance. The method includes securing a belt around the abdomen of a user. The belt comprises an extensible belt portion. The method further includes measuring the length of the extensible belt portion as the user breathes. The method further includes converting the measurement of the length into an electronic signal. The method further includes transmitting the electronic signal to a remote device. The method further includes computing a breathing curve from the electronic signal. The method further includes displaying the breathing curve in a graphical form.

### BRIEF DESCRIPTION OF THE DRAWIGS

FIG. 1: Abdominal breathing belt with a resistive fabric sensor.

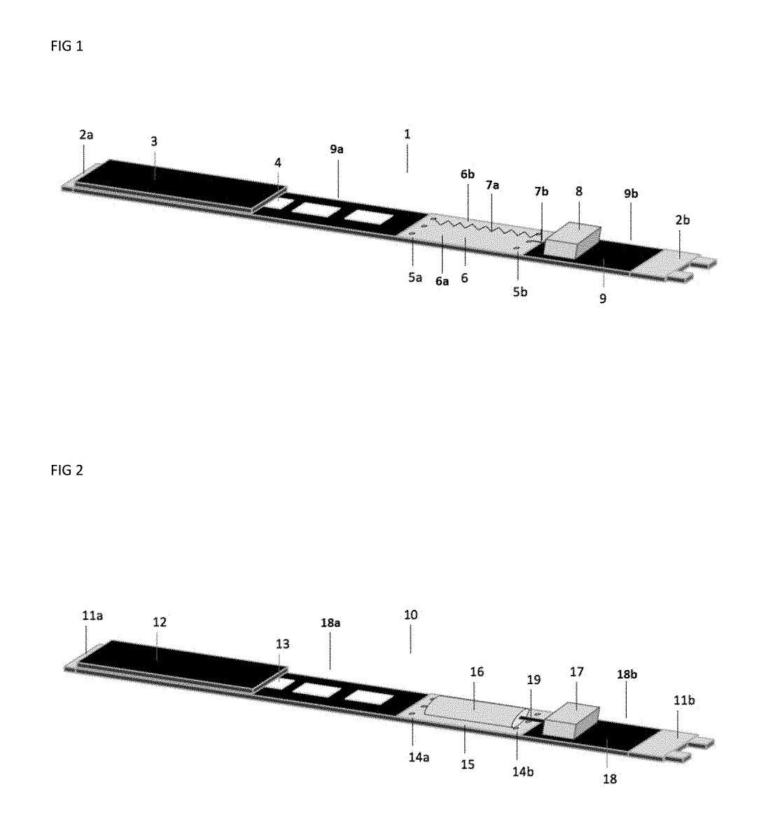

FIG. 2: Abdominal breathing belt with a string potentiometer or digital encoder sensor.

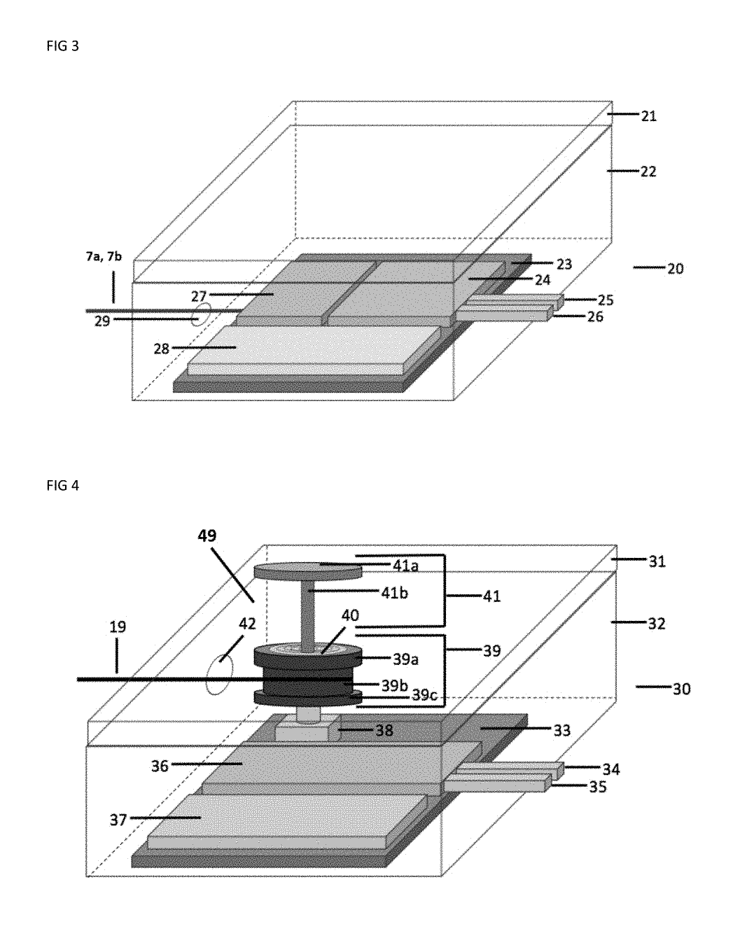

FIG. 3: Electronics box for an abdominal breathing belt with a resistive fabric sensor.

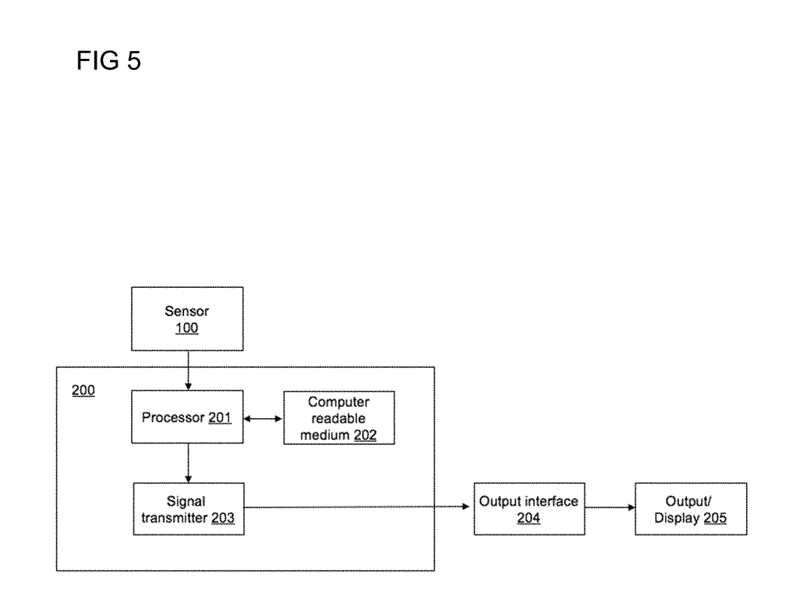

FIG. 4: Electronics box for an abdominal breathing belt with a string potentiometer or digital encoder sensor.

FIG. 5: General flow of data transmission.

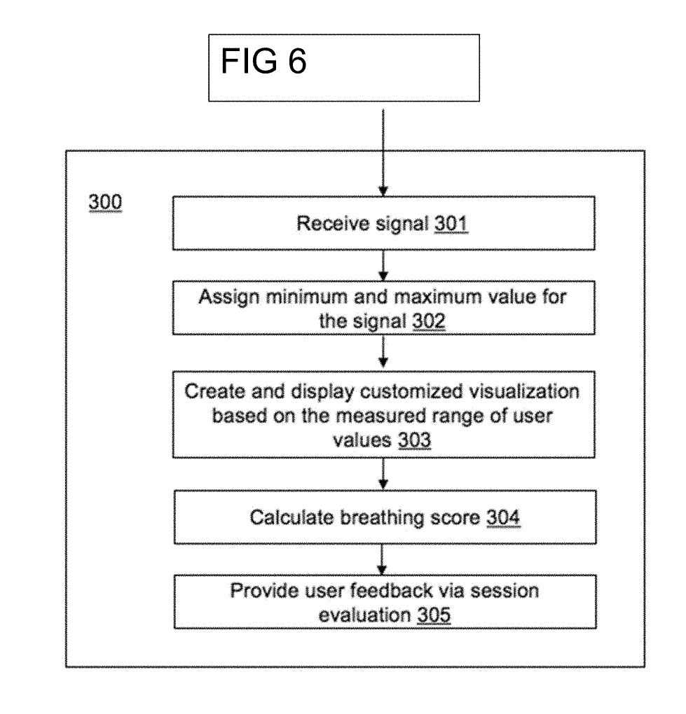

FIG. 6: General flow of data through one type of output interface, such as a phone application.

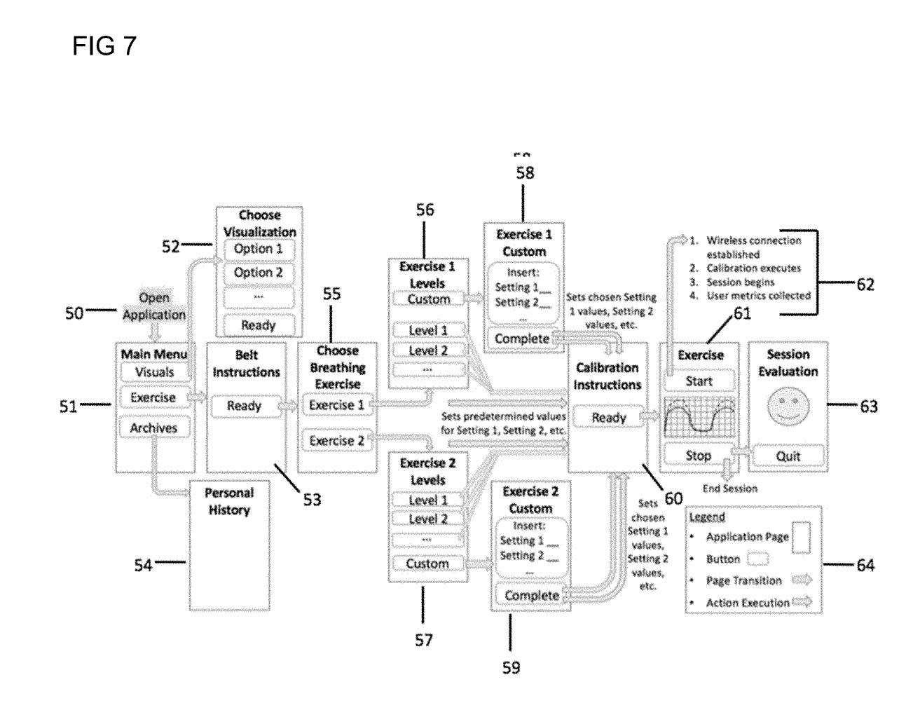

FIG. 7: Schematic describing one illustrative embodiment of a possible phone application, showing the flow of user options.

### DETAILED DESCRIPTION

The abdominal expansion measuring device (1) may include a belt, a portion of which, may be comprised of an electrically conductive extensible fabric that generates a measurement based on resistance changes that are a function of the user's abdominal expansion and the tensile properties of the fabric. In another embodiment, the abdominal expansion measuring device (10) may include a potentiometer that generates a measurement based on a resistance change that is a function of the user's abdominal expansion. In another embodiment, the abdominal expansion measuring device (10) may include a digital encoder that generates a digital change in position as a function of the user's abdominal expansion.

Referring to FIG. 1, a view of one embodiment of a wearable biofeedback belt device is shown in accordance with an illustrative embodiment. The abdominal expansion measuring device (1) comprises a belt (9) that is comprised of a first inextensible belt portion (9a), a second inextensible belt portion (9b), and an extensible belt portion (6) that connects either permanently or removably to the first inextensible belt portion and the second inextensible belt portions. The belt (9) is configured to fit around the abdomen of a user. Attached to each end of the belt (9) is part of a fastening mechanism (2a, 2b). The first side of the fastening mechanism (2a) is affixed to one end of the belt (9) while the second side of the fastening mechanism (2b) is affixed to the other end of the belt (9). The fastening mechanism can be a clip, a snap, two complementary fastening materials, or a variety of other fastening means. The abdominal expansion measuring device (1) may be adjustable to fit users of different sizes. The belt (9) may have complementary securing means (4) located on appropriate portions of the belt (9). The complementary securing means (4) are configured to secure an inextensible strap (3) to the belt (9) after the user adjusts the belt to the desired length. The complementary securing means (4) can be snaps, fastening material, magnets, or a variety of other complementary fastening means.

The extensible belt portion (6) may be comprised of a patch, or one or more strips, of extensible, or stretchable, fabric (6a). The extensible belt portion (6) is extensible so that there is “give” in the belt (9). A plurality of fasteners (5a, 5b) can be placed across the width of the extensible belt portion (6) on each of its ends. A first set of fasteners (5a) can be placed along one end of the extensible belt portion (6) widthwise, while a second set of fasteners (5b) can be placed along the opposite end of the extensible belt portion (6) widthwise. The fasteners (5a, 5b) may be electrically conductive. The fasteners (5a, 5b) can be snaps, or a variety of other fastening means. The fasteners (5a, 5b) may serve as a physical connection between the extensible belt portion (6) and the first and second inextensible belt portions (9a, 9b). The fasteners (5a 5b) may permanently or removable connect the extensible belt portion (6) to the first and second inextensible belt portions (9a, 9b). It may be desirable to have the extensible belt portion (6) be removable so that it can be washed or replaced. When the abdominal expansion measuring device (1) is secured around the abdomen of the user, the extensible belt portion (6) expands and contracts with the expansion and contraction of the user's abdomen. The abdominal expansion measuring device (1) should be secured to the user's abdomen so that there is a minimum non-zero tension in the extensible belt portion (6) when the user's abdomen is at full contraction.

The segment of extensible fabric (6a) may be coated with an electrically conductive coating (6b). The resistance of the electrically resistive coating (6b) may change with the extension of the fabric strips (6a). The length, and thereby the extension, of the extensible belt portion (6) can be measured by measuring the resistance across all or a portion of the electrically resistive coating (6b). Two electrical leads (7a, 7b) may be connected to the electrically resistive coating at different locations on the extensible belt portion (6). The electrical leads (7a, 7b) can be a wire, conductive thread, or a variety of other electrically conductive materials. The electrical leads may be secured to the extensible belt portion (6) in a zig-zag pattern, or other extensible pattern, so that the electrical leads (7a, 7b) do not interfere with the expansion or contraction of the extensible belt portion (6). The fasteners (5a, 5b), which may be electrically conductive, may be used to secure the electrical leads (7a, 7b) to the electrically conductive coating (6b). The full width of fasteners (5a, 5b) may be used to acquire a signal across the full width of the extensible belt portion (6) to produce a clean electrical signal. The other ends of the electrical leads (7a,7b) may be connected to the belt expansion measuring device (8). The belt expansion measuring device (8) is configured to produce an electronic signal that corresponds to, and thereby measures, the length of the extensible belt portion (6) or the belt. The length of the extensible belt portion (6) can be correlated to the length of the belt (9) by adding an offset to the measurement of the length of the extensible belt portion (6) corresponding to the lengths of the first inextensible belt portion (9a) and the second inextensible belt portion (9b). The belt expansion measuring device (8) may take more than one measurement of the length of the extensible belt portion (6) during the user's breathing cycle, so that a change in the length of the extensible belt portion (6) can be determined. Thereby, the change in the extension, or expansion, of the user's abdomen can be determined.

The belt extension measuring device (8) comprises an electronics box (20). The electronics box (20) can be permanently or removably attached to the belt (9). The electrical leads (7a,7b) may be connected to a microcontroller (23) within the electronics box (20). The electrical signal from the electrical leads (7a,7b), and thereby from the extensible belt portion (6), is transferred to a microcontroller (23) within the electronics box (20), which converts raw values into units that can be processed by a signal transmitter (27), which also may be within the electronics box (20). An output interface (204) such as a cellular phone or a computer is configured to receive a signal from the electronics box (8). The signal transmitter (27) may support wireless communication using various transmission media and may include, for example, a Bluetooth® connection, WiFi connection, radio connection, or other transmission media.

FIG. 3 shows a first illustrative embodiment of the electronics box (20). The electronics box (20) may have a top portion (21) and a bottom portion (22). The electrical leads (7a, 7b,) attaching to the extensible belt portion (6) may enter the electronics box (20) through an opening (29). The electrical leads (7a,7b) may interface with a microcontroller (23) within the electronics box (20). The microcontroller (23) may interface with a signal transmitter (27) that transmits information to an output interface (204) wirelessly. The microcontroller (23) may be responsible for collecting data corresponding to the extension of the belt (9), and thereby the expansion of the user's abdomen, as the user inhales and exhales. There may be an optional processing module (28) that may be used to modify the electrical signal from the electrical leads (7a,7b). The processing module (28) may provide additional processing of the electrical signal. The processing module (28) may also include an optional filter that can be hardware, software, etc. to help produce a cleaner signal. The microcontroller (23) may be powered by an internal power source (24). The power source (24) can be a battery, a chemical power source, or a variety of other power sources. There may be an optional charging port (25), which may connect to the power source (24) and may interface with a standard cable to allow the user to charge the device. There may be an optional switch (26) that may be positioned to interact with the power source. The switch (26) may connect the optional charging port (25) to the power source (24) when “off”, allowing the power source (24) to be recharged if it is electrical.

Referencing FIG. 2, another illustrative embodiment of the abdominal expansion measuring device (10). Like the first embodiment of the abdominal expansion measuring device (1), the second embodiment of the abdominal expansion measuring device (10) comprises a belt (18) that is comprised of a first inextensible belt portion (18a), a second inextensible belt portion (18b), and an extensible belt portion (15), two parts of a fastening mechanism (11a, 11b), complementary securing means (13) located appropriately on the belt (18) to hold excess fabric (12) in place after user adjustment (if the belt is adjustable), and belt extension measuring device (17). The extensible belt portion (15) may be attached either permanently or removably to the belt (18). The extensible belt portion (15) may host a plurality of fasteners (14a, 14b) that span opposite sides of the extensible belt portion (15) widthwise. In this exemplary embodiment, the plurality of fasteners (14a, 14b) provide a physical connection between the extensible belt portion (15) and the first and second inextensible belt portions (18a, 18b)

A string, cord, or filament (19) may connect at one end to a location on the extensible belt portion (15) and at the other end to a positional transducer (49) located within the electronics box (30). The string (19) may connect to a location on the extensible belt portion (15) that is at the end of the extensible belt portion furthest away from the electronics box (30), or at some other appropriate location on the extensible belt portion (15). The string (19) may connect to a location on the inextensible belt portion (18a) on the opposite side of the extensible belt portion (15) compared to the electronics box (30) location. The string (19) may protrude from the electronics box (30). The string (19) may be a cable, wire, thread, or a variety of other materials. The string (19) is preferably inextensible. The string (19) can connect permanently or removably to the extensible connecting fabric (15). The string (19) will extend and retract as the extensible connecting fabric (15) extends and retracts with the user's breathing. The string (19) may be enclosed in an optional sleeve (16) that guides the string (19) and protects it from damage. The protective sleeve (16) may be permanently or removably attached to the extensible connecting fabric (15) or to the inextensible belt portion (18a) on the opposite side of the extensible belt portion (15) compared to the electronics box (30) location. The sleeve may be secured to the extensible belt portion (15) or to the inextensible belt portion (18a) by snaps or other similar type of fasteners.

FIG. 4, shows a second illustrative embodiment of the electronics box (30). The electronics box (30) can have a top portion (31) and a bottom portion (32); inside of the electronics box (30) can be a microcontroller (33), an optional charging port (34), an optional switch (35), a signal transmitter (36), and an internal power source (37) that function similarly to their counterparts described in the above discussion of FIG. 3. This embodiment of the electronics box (30) comprises a positional transducer (49). The positional transducer (49) comprises the filament, or string (19), a rotating spool (39), and a sensor (38). The positional transducer (49) converts the position of an element, such as the end of the string in this case, into an electronic signal. The string (19) interfaces with a rotating spool (39). The spool (39) interfaces with a sensor (38) such that the rotation of the spool (39) is converted to an electronic signal that is read by the microprocessor (33), to which the sensor (38) is connected. Embodiments of the sensor (38) may include a rotational potentiometer, an encoder (for example a digital encoder), or a similar sensing mechanism. A rotational potentiometer is a device containing a variable resistor that converts rotational position into a resistance value and transmits that information to the microcontroller (33). An encoder, or digital encoder, is a device that creates a signal based on the relative rotational position of the spool (39) and transmits that information to the microcontroller (33). The belt expansion measuring device (17) is configured to produce an electronic signal that corresponds to, and thereby measures, the rotational position of the spool (39). The rotational position of the spool (39) can be correlated to the distance between the spool (39) and the end of the filament secured to the belt (9); i.e. the length of the filament (19) between the first end of the filament (19) and the spool (39), as well as to the length of the extensible belt portion (15). The length of the extensible belt portion (15) can be correlated to the length of the belt (18). The belt expansion measuring device (17) may take more than one measurement of the length of the extensible belt portion (15) during the user's breathing cycle, so that a change in the length of the extensible belt portion (15) can be determined. Thereby, the change in the extension, or expansion, of the user's abdomen can be determined.

Both the potentiometer and encoder may be shaped very similarly, and therefore the following descriptions are valid for either sensor type. A string (19) attaches to the extensible belt portion (15) and may enter the electronics box (30) through an opening (42). The string (19) will wrap around a spool (39), likely though not limited to the spool's middle portion (39b). The spool (39) may have a top cap (39a) and a bottom cap (39b) to prevent the string (19) from falling off the spool (39). Interfacing with the spool (39) is a spring (40). The spring (40) may be oriented and pretensioned such that it opposes the rotation of the spool (39), or resists the tension in the string. The spool (39) also interfaces with the sensor (38) beneath it. The sensor (38) and the spool (39) can interface via complementary notches, adhesives, or a variety of other interfacing techniques. When the spool (39) rotates, the sensor (38) rotates. For example, as the user breathes in, their abdomen expands, the extensible portion of the belt (15) extends, the spool (39) may rotate to allow more string (19) to exit through the opening (42) of the electronics box (30), the sensor (38) also rotates the same amount as the spool (39), and the spring (40) may be pulled to a state of increased tension. When the user relaxes, their abdomen contracts, the extensible portion of the belt (15) retracts, and the spring (40) can recoil back to a state of lesser tension. Therefore, the spring (40) rotates the spool (39) and the sensor (38) in a direction opposite of the rotation experienced during user inhalation.

The microcontroller (33) is responsible for collecting abdominal expansion data as the user inhales and exhales. The microcontroller (33) may contain software code that converts sensor values into values that the signal transmitter (36), for example a Bluetooth® chip or other signal transmitter, can read. For example, the microcontroller (33) may convert resistance values to voltages.

A spring turning tool (41) may be used to hold the spring (40) in place. Further, rotating such a spring turning tool (41) may be a method of adjustably pretensioning the spring (40). The spring (40) must be held in place by a mechanism that the spring (40) can rotate with respect to, otherwise the spring (40) will not gain and lose tension as the user breathes in and out, and the spool (39) might not be pulled back to its initial state as the extensible belt portion (15) contracts. If a spring turning tool (41) or a similar mechanism is not used, the walls of the electronics box (30) can be used to hold the spring (40) in place, or another mechanism can be used. The spring turning tool (41) is positioned such that it holds the center of the spring (40) stationary without pushing the spring (40) forcefully into the cap of the spool (39a), which may cause friction that could disrupt sensor (38) movement. A variety of securing methods may be used to fix the position of the spring turning tool (41) with respect to the walls of the electronics box (30). The spring turning tool (41) may have a top portion (41a) that interfaces with the walls of the electronics box (30) and a bottom portion (41b) that interfaces with the spring (40).

Referring to FIG. 5, a flow of data transmission is shown in accordance with an illustrative embodiment. The order of presentation of the operations of FIG. 5 is not intended to be limiting. Although some of the operational flows are presented in sequence, the various operations may be performed in various repetitions, concurrently, and/or in other orders than those that are illustrated. Sensor (100) may be housed within a housing (200) for protection from the environment. The electronics within the housing (200) may include a processor (201), a computer-readable medium (202), and a signal transmitter (203). The signal from the signal transmitter (203) may interface with an output interface (204) and possibly with an output or display (205). The processor (201) receives a signal from the sensor (100) that varies as a function of the user's abdominal expansion and contraction. The output interface (204) provides a means of outputting information received from the signal transmitter (203). For example, the output interface (204) may interface with various outputs or displays (205) including, but not limited to, a visual display, a speaker, a light, a vibrating mechanism, etc. In one embodiment, the output display (205) is a graphical display of the user's breathing pattern, which may include the amplitude and/or frequency of abdominal expansion in real time.

The computer-readable medium (202) is an electronic holding place or storage for information so the information can be accessed by the processor (201) as understood by those skilled in the art. The computer-readable medium (202) can include, but is not limited to, any type of random access memory (RAM), any type of read only memory (ROM), any type of flash memory, etc. such as magnetic storage devices (e.g., hard disk, floppy disk, magnetic strips, etc.), optical disks (e.g., compact disc (CD), digital versatile disc (DVD), etc.), smart cards, flash memory devices, etc. The output interface (204) may have one or more computer-readable media that use the same or a different memory media technology. For example, the computer-readable medium (202) may include different types of computer-readable media that may be organized hierarchically to provide efficient access to the data stored therein as understood by a person of skill in the art. As an example, a cache may be implemented in a smaller, faster memory that stores copies of data from the most frequently/recently accessed main memory locations to reduce an access latency. The output interface (204) also may have one or more drives that support the loading of a memory media such as a CD, DVD, an external hard drive, etc.

The processor (201) executes instructions as understood by those skilled in the art. The instructions may be carried out by a special purpose computer, logic circuits, hardware circuits, or other methods. The processor (201) may be implemented in hardware and/or firmware. The processor (201) executes an instruction, meaning it performs/controls the operations called for by that instruction. The term “execution” is the process of running an application or the carrying out of the operation called for by an instruction. The instructions may be written using one or more programming language, scripting language, assembly language, etc. The processor (201) operably couples with the computer-readable medium (202) and the output interface (204) to receive, to send, and to process information. The processor (201) may retrieve a set of instructions from a permanent memory device and copy the instructions in an executable form to a temporary memory device that is generally, though not limited to, some form of RAM. The output interface (204) may include a plurality of processors that use the same or a different processing technology.

In reference to FIG. 6, an illustrative embodiment is shown where the output interface (204) is a phone application (300). Some or all of the operations described herein may be embodied in the breathing application (300). The application first receives a signal (301) from the abdominal expansion measuring device (1) that varies with the expansion of a user's abdomen. The application may then perform operations associated with producing a user-specific breathing display, for example a breathing curve, based on a signal transmitted from the abdominal expansion measuring device (1). In a calibration operation (302), a minimum value and a maximum value are used to create a relative scale, for example, a scale having a value ranging from 0 to 10. In one embodiment, the user may be instructed to expand their abdomen as far outward as possible, creating a maximum signal value correlated to the upper range of the calibration scale (for example, a value of “10”). The user may also be instructed to contract their abdomen as far inward as possible, creating a minimum signal value correlated to the lower range of the calibration scale (for example, a value of “0”). In some embodiments of the invention, this calibration operation (302) is based on the signal generated by a time period (e.g., fifteen seconds) of the user's breathing. In a display operation (303), a graphical interface is created using values in between the calibrated maximum and minimum values. Such an interface may be, but is not limited to, a sine wave or various other gamified visuals. For example, a circular ball may expand in diameter as the user inhales and decrease in diameter as the user exhales. In another possible embodiment, movement of the user's abdomen may cause movement of a graphic. For instance, user inhalation may correspond to upward movement of an image while exhalation may correspond to downward movement of an image. The user can voluntarily coordinate their abdominal expansion, or breathing, to align their breathing with the personalized display and/or to manipulate the personalized display in an ideal way; for instance, to comply with the rules of a game. In an optional assessment operation (304), the user's breathing score is calculated. What constitutes a score, as well as the score calculation method, may vary based on the graphical visualization used. For example, an assessment operation (304) may involve computing the difference between an ideal breathing curve generated by the application (300) software and the user's measured breathing curve, which may be superimposed over the ideal breathing curve. A user may receive a higher score if there is a smaller difference between the ideal breathing curve and the user-generated measured breathing curve. An ideal breathing curve may represent a pre-set breathing target demonstrating exemplary breathing of a particular type. For instance, an ideal breathing curve for athletes of a specific sport may have a specific amplitude and rate. Similarly, an ideal breathing curve for specific wind-instrument musicians may have a different specific amplitude and breathing rate. Different breathing rates and amplitudes can be employed to help users reach specific breathing goals applied to specific contexts. If the optional assessment operation (304) is carried out, an optional feedback operation (305) may also execute, allowing the user to see an evaluation of their performance based on the calculated outcomes of the assessment operation (304).

Each of these operations can be carried out iteratively in real-time. Creating and displaying a customized visualization (303) based on the measured range of user values (302) may be implemented in software (comprised of computer-readable and/or computer-executable instructions), stored in computer-readable medium (202), and accessible by the processor (201) for execution of the instructions that embody the operations of the phone application (300). The application (300) may be written using one or more programming languages, assembly languages, scripting languages, etc. The application (300) may be implemented as a mobile application, a Web application, etc. For example, the application (300) may be configured to receive hypertext transport protocol (HTTP) requests and to send HTTP responses.

In reference to FIG. 7, an illustrative example of one possible embodiment of an output interface (204) is provided, where the output interface (204) is a phone and the output display (205) is one example of a possible phone application. When operating the phone application (300), the user may first see a brief video. The content of the video may include, but is not limited to, an explanation of the benefits of abdominal breathing and/or a description of how the application (300) works. Alternatively, the user may first open the application (50) and view a main menu (51) with a variety of preset options. These options may include, but are not limited to, the user's ability to choose preferred visuals, the choice to begin a new breathing exercise session, and the ability to view assessments of past sessions that are stored in a personalized archive (54). If the user chooses to pick their graphical settings, they may be taken to a visualizations page (52). A visualizations page (52) may contain a variety of pre-set graphical displays that the user can choose from. Such graphical displays may include, but are not limited to, a sine graph of the user's abdominal expansion overlaid atop an idealized sine wave, a ball that swells and contracts as the user extends and contracts their abdomen, an animal figure that moves toward treasure or treats as the user expands their abdomen, a ball that elevates to an area of the screen worth more points as the user expands their abdomen, or a vast array of other possible graphical interfaces. Alternatively, the user may have the choice between visual and non-visual methods of feedback. For instance, a user may choose to receive verbal feedback, tactile feedback, or other forms of feedback instead of receiving graphical feedback. Other forms of feedback used (for example, verbal feedback, tactile feedback, etc.) may also include a variety of preset options available to the user, and/or the ability for the user to customize their feedback as they so choose.

Alternatively, the user may opt not to choose a graphical interface and may instead choose to begin a breathing exercise session. In this case, they may be given instructions regarding how to properly put on the abdominal expansion measuring device (53) and they may be directed to a breathing exercise page (55). On the breathing exercise page (55), the user may be prompted to select a breathing style from a series of preset options. Preset breathing options may include, but are not limited to, stress relief exercises, general abdominal breathing exercises, Hara breathing exercises, musical instrument-specific exercises (ex: tuba, trombone, clarinet, etc.), sport specific exercises (ex: swimming, running, dance, etc.), illness specific exercises (ex: emphysema, generalized anxiety disorder, etc.); as well as a wide variety of other options. Once a user has selected an exercise, they may be redirected to an exercise-specific screen (56, 57) where they can choose from preset levels that may vary in their duration, difficulty, or a variety of other distinguishing factors. There may also be an option for the user to bypass the preset levels and customize their session instead. To customize their settings, a user may open an exercise-specific customization page (58, 59) and choose specific values for available adjustable settings. Adjustable settings may include choosing the duration of a session, the desired breathing rate during a session, the desired maximal abdominal expansion achieved during a session, or a variety of other variables.

The application may also contain a calibration feature (60) whose purpose is to gauge the user's personal maximum and minimum abdominal positions. The graphical representations and user metrics may be based on this user-specific baseline. Calibration ensures that each user's breathing is assessed based on their personal abilities. If calibration is used, the user may begin a breathing session after calibration is complete. The breathing exercise display page (61) can give the user real-time biofeedback assessing their physical abdominal expansion. The exercise display page (61) may display information in the user's desired graphical format, if the user chose a specific interface. For a breathing exercise session to be successful, the exemplary application described herein may check for various requirements to be met (62). For example, an application may require an established wireless connection, proper calibration (if a calibration feature (60) is used), the starting of a session, and the collection of user metrics. User metrics may include, but are not limited to, the number of “points” a user acquires while using a gamified version of the application, how closely the user's sine graph of abdominal movement mirrors a sine wave of ideal breathing, how long a user is able to consecutively match their breathing pattern within a certain percent error to an ideal breathing patter, objective measurements of the user's maximum amplitude (maximum abdominal expansion achieved), objective measurements of user's breathing rate, as well as a wide variety of other user metrics. User metrics may be used to determine the success of a single session and to gauge improvement over time. Such metrics can be stored in a personalized archive (54) for the user to revisit when they wish. Once the user chooses to end a session or the session duration terminates, the user may view a session evaluation (63). A session evaluation (63) may use the user metrics collected during the breathing session to give the user an objective assessment of their performance.

1. A wearable device comprising:
a belt configured to fit around the abdomen of a user;
a belt expansion measuring device operably coupled to the belt and configured to generate a signal that varies as a function of the expansion and contraction of the user's abdomen during breathing, the belt expansion measuring device comprising a signal transmitter; and
a remote device configured to receive the signal from the belt expansion measuring device, to compute a breathing display from the signal, and to display the breathing display curve in graphical form.

2. A method of generating a breathing curve using the wearable device of claim 1, the method comprising placing the belt around the abdomen of a user, wherein the belt expansion measuring device generates a signal that varies as a function of the expansion and contraction of the user's abdomen during breathing and transmits the signal to the remote device and the remote device receives the signal from the belt expansion measuring device, computes the user's breathing curve from the signal, and to displays the breathing curve in graphical form.

3. A belt comprising:
a first inextensible belt portion;
a second inextensible belt portion;
an extensible belt portion disposed between the first inextensible belt portion and the second inextensible belt portion; and
a belt expansion measuring device configured to measure a length of the extensible belt portion and produce an electronic signal that corresponds to the length of the extensible belt portion.

4. The belt of claim 3,
wherein the belt expansion measuring device is attached to one of the first inextensible belt portion or the second inextensible belt portion.

5. The belt of claim 3,
wherein the extensible belt portion comprises:
an extensible fabric;
an electrically conductive coating on the extensible fabric, the electrically conductive coating configured to change resistance as the extensible fabric changes length;
a first electrical lead electrically connected to the electrically conductive coating at a first position on the extensible belt portion; and
a second electrical lead electrically connected to the electrically conductive coating at a second position on the extensible belt portion,
wherein the belt expansion measuring device is configured to measure resistance of a portion of the electrically conductive coating between the first position and the second position.

6. The belt of claim 3,
wherein the belt expansion measuring device comprises:
a positional transducer operatively attached to the first inextensible belt portion; and
a filament having a first end and a second end, the first end operatively attached to a location on either the extensible belt portion or the second inextensible belt portion and the second end attached to the positional transducer,
wherein the belt expansion measuring device is configured to measure the length of the filament between the first end and the positional transducer.

7. The belt of claim 6, wherein the positional transducer comprises:
a microprocessor;
a spool; and
a sensor connected to the spool and to the microprocessor, the sensor configured
to produce an electronic signal corresponding to a rotational position of the spool, wherein the filament is connected to the spool so that movement of the filament causes rotation of the spool.

8. The belt of claim 7,
wherein the positional transducer further comprises a spring, wherein the spring resists tension in the filament.

9. The belt of claim 7,
wherein the sensor is a rotational encoder.

10. The belt of claim 7,
wherein the sensor is a rotational potentiometer.

11. The belt of claim 3,
wherein the belt expansion measuring device comprises a transmitter configured to transmit the electronic signal to a remote device.

12. The belt of claim 3,
wherein the belt expansion measuring device further comprises a power supply.

13. A method of monitoring breathing performance, the method comprising the steps of:
securing a belt around the abdomen of a user, the belt comprising an extensible belt portion;
measuring the length of the extensible belt portion as the user breathes;
converting the measurement of the length into an electronic signal;
transmitting the electronic signal to a remote device;
computing a breathing display curve from the electronic signal, and
displaying the breathing display curve in a graphical form.
